(19) United States
(12) Patent Application Publication
(10) Pub. No.: US 2014/0193876 A1
(43) Pub. Date: Jul. 10, 2014

(54) METHODS OF PURIFICATION OF NATIVE OR MUTANT FORMS OF DIPHTHERIA TOXIN

(71) Applicants: Aaron R. Goerke, Wayne, PA (US); Thomas Svab, Plains, PA (US); Patrick McHugh, Perkasie, PA (US); Kristin Valente, Plymouth Meeting, PA (US)

(72) Inventors: Aaron R. Goerke, Wayne, PA (US); Thomas Svab, Plains, PA (US); Patrick McHugh, Perkasie, PA (US); Kristin Valente, Plymouth Meeting, PA (US)

(21) Appl. No.: 14/125,695

(22) PCT Filed: Jun. 8, 2012
(86) PCT No.: PCT/US2012/041444

§ 371 Date: Mar. 13, 2014

Related U.S. Application Data
(60) Provisional application No. 61/496,276, filed on Jun. 13, 2011.

(51) Int. Cl. C12N 9/10

(52) U.S. Cl. CPC C12N 9/1077
USPC 435/193

(57) ABSTRACT

The present invention relates to the use of hydroxyapatite chromatography and multimodal chromatography, for punfication of diphtheria toxin, or a mutant form thereof, from a mixture, for example, a host cell fermentation mixture containing impurities such as host cell proteins and DNA. This invention further relates to the integration of such a method into a multi-step procedure with other fractionation methods for purification of diphtheria toxin suitable for in vitro and in vivo applications.

### CROSS-REFERENCE TO RELATED APPLICATIONS

Not Applicable

### FIELD OF THE INVENTION

The present invention relates to methods for the purification of native or mutant forms of diphtheria toxin using hydroxyapatite chromatography and multimodal chromatography. In certain embodiments, the mutant form of diphtheria toxin is CRM₁₉₇.

### BACKGROUND OF THE INVENTION

Diphtheria toxin is a proteinaceous toxin which is synthesized and secreted by toxigenic strains of Corynebacterium diphtheriae. Diphtheria toxin and its mutant forms have found applications in both vaccines, as a carrier protein, and anticancer drugs, as a targeted therapy. Formaldehyde-inactivated diphtheria toxin has been used for vaccination against C. diphtheriae since the 1920's. Conjugate vaccines using mutant forms of diphtheria toxin began becoming widely available in the 1980s. See Shinefield, 2010, Vaccine 28:4335-4339. The ability of diphtheria toxin to stimulate T-cell immunity makes it an attractive carrier protein for T-cell independent antigens (e.g. polysaccharides). Diphtheria toxin is also used as a cancer therapy by conjugating a ligand that specifically targets overexpressed surface proteins on tumor cells. See Michl et al., 2004, Curr Cancer Drug Targets, 4:689-702; and Potala et al., 2008, Drug Discovery Today 13:807-815.

Mutant forms of diphtheria toxin are highly desirable in both vaccines and anticancer agents. In vaccines, diphtheria toxin is typically mutated to reduce toxicity. Such mutations can cause loss of ADP-ribosylation activity which, in the native toxin, blocks protein synthesis. In anticancer agents, diphtheria toxin is typically mutated to eliminate binding to its native receptor on normal cells. See Potala et al., 2008, Drug Discovery Today 13:807-815.

In vaccine applications, CRM₁₉₇ is the most widely used mutant of diphtheria toxin. CRM₁₉₇ is produced by a mutant strain of C. diphtheriae differing from diphtheria toxin in the presence of a glutamic acid instead of a glycine in position 52 and is essentially non toxic. See Uchida et al., 1973, J Biol Chem 248:3838-3844. CRM₁₉₇ is currently widely used as a carrier for vaccines for pediatric use. See Shinefield, 2010, Vaccine 28:4335-4339.

Methods for purification of diphtheria toxin, and mutant forms of diphtheria toxin, that have been used include affinity chromatography (Cukor et al., 1974, Biotech and Bioeng 16:925-931; and Antoni et al., 1983, Experientia 39:885-886), anion-exchange in combination with hydrophobic chromatography (Rappuoli et al., 1983, J. Chromatog 268:543-548), and tangential flow filtration (Sundaran et al., 2002, J Biosci and Bioeng 94:93-98).

For clinical use, large quantities of mutant diphtheria toxin are needed. There are however problems in producing diphtheria toxin from diphtheria toxin producing strains of C. diphtheriae, and moreover, difficulties have been encountered in scaling up laboratory scale fermentation conditions to produce sufficient quantities of diphtheria toxin, and in particular mutant forms of diphtheria toxin, for therapeutic use. Thus, there are problems in obtaining diphtheria toxin in sufficient yield and purity and large scale production tends to be inefficient. For instance, low pH induces conformational changes and promotes aggregation, making the use of typical cation exchangers (and lower pHs) difficult. Proteolytic cleavage (by host proteases or autocatalytically) also occurs frequently for most toxins causing heterogeneity. Heterogeneity can be detected in the form of, for example, product isoforms, product variants, product fragments, or glycosylation patterns. These product forms must be removed which remains a challenging feature of toxin purification in general and for diphtheria toxin specifically. These difficulties need to be overcome in order to be able to meet the current needs with pediatric vaccines and to exploit the promise of targeted anticancer drugs.

What are needed are purification methods that provide for efficient purification and high yields of diphtheria toxin.

Citation or identification of any reference in this section or any other section of this application shall not be construed as an indication that such reference is available as prior art to the present invention.

### SUMMARY OF THE INVENTION

The present invention relates to methods of purifying mutant diphtheria toxin, and mutant forms thereof, for example CRM₁₉₇, from intact cells which provide for high purity and yield. The critical step in this process has been found to be the removal of endotoxin and residual proteins by the use of a hydroxyapatite resin. A multimodal chromatography step is also performed. In one aspect, use of a multimodal chromatography resin immediately follows the hydroxyapatite resin. In another aspect, use of a multimodal chromatography resin immediately precedes the hydroxyapatite resin.

Thus, in one embodiment, the present invention relates to a method of purifying diptheria toxin, or a mutant form thereof, from a mixture containing the diptheria toxin, or mutant form thereof, comprising:

a) contacting the mixture with a first separation agent under conditions such that the diphtheria toxin, or mutant form thereof, binds to the first separation agent;

b) eluting the diphtheria toxin, or mutant form thereof, from the first separation agent;

c) contacting the eluted material obtained from step a) with a second separation agent under conditions such that the diphtheria toxin, or mutant form thereof, binds to the second separation agent; and

d) eluting said diptheria toxin, or mutant form thereof, from the second separation agent;

wherein either 1) the first separation agent is hydroxyapatite and the second separation agent is a multimodal resin; or 2) the first separation agent is a multimodal resin and the second separation agent is hydroxyapatite; and when the first separation agent or second separation agent is hydroxyapatite, prior to eluting from the hydroxyapatite, the hydroxyapatite undergoes a washing step under conditions such that impurities are removed.

In a certain aspect, the first separation agent is hydroxyapatite and the second separation agent is a multimodal resin.

In certain aspects, the washing of the hydroxyapatite is with a wash solution comprising from 0.01 to 1.0 M potassium chloride or sodium chloride at a pH from 6.5 to 8.0. The wash buffer may further comprise 0.1 to 20 mM potassium phosphate or sodium phosphate.

In certain aspects, the eluting from the hydroxyapatite comprises i) step elution with an elution buffer comprising ≧about 30 mM potassium chloride or sodium chloride or about ≧15 mM potassium phosphate or sodium phosphate, ii) gradient elution comprising from about 10 to about 25 mM potassium phosphate or sodium phosphate or from about 100 mM to 2 M potassium chloride or sodium chloride; or iii) a pH change of ≧0.3 pH units.

In certain aspects, the mixture contacted with the multimodal resin comprises Tris, MES, MOPS, HEPES, phosphate (e.g., potassium), chloride (e.g., potassium or sodium), or phosphate (e.g., sodium).

In certain aspects, the mixture is eluted from the multimodal resin by i) step elution with an elution buffer comprising ≧about 125 mM potassium chloride or sodium chloride at pH 6.8 to 9.5; ii) gradient elution comprising from about 0.2 to about 0.3 M sodium chloride, potassium chloride, sodium sulfate, ammonium sulfate, or potassium chloride; iii) a pH change of ≧0.5 pH units within a pH range of 6.5 to 9.5; or iv) a temperature change of ≧1° C. within a temperature of 2 to 30° C.

In certain aspects of the invention, the multimodal resin contains ligands which comprise a charged part and a hydrophobic part. In certain aspects, the charged part is a negatively charged part, for example, an anionic carboxylate group or anionic sulfo group for cation exchange. An example of such a multimodal resin is Capto-MMC™. In other aspects, the charged part is a positively charged part, e.g., an amino group. An example of such a multimodal resin is Capto Adhere™.

In certain aspects of the invention, the mixture contacted with the multimodal resin comprises EDTA or a protease inhibitor. In other aspects of the invention, elution from the multimodal resin occurs in the presence of EDTA or a protease inhibitor.

In different embodiments, application of the mixture to hydroxyapatite or a multimodal resin, whichever comes first, may be preceded by one or more of the following: centrifugation, flocculation, clarification, or anion-exchange chromatography. In certain aspects of the invention, the mixture is subject to two or three passes on anion-exchange chromatography. In certain aspects of the invention, application of the mixture is preceded by centrifugation, flocculation, clarification, and anion-exchange chromatography. Clarification may be through centrifugation and depth filtration.

In certain embodiments of the invention, the beginning mixture is a fermentation culture of host cells. In certain aspects of this embodiment, the cultured host cells are harvested and osmotically shocked to release diphtheria toxin, or a mutant form thereof, from the periplasm. In other aspects, the fermentation cells are recovered by centrifugation or microfiltration.

In other embodiments of the invention, the beginning mixture is obtained from a cell-free production system.

In certain embodiments, the mixture containing diphtheria toxin, or a mutant form thereof, is subject to one or more of the following after the contacting and eluting from the hydroxyapatite resin and the contacting and eluting from the multimodal resin: centrifugation, ultrafiltration, microfiltration, filtration and anion-exchange membrane chromatography. In certain aspects, the mixture containing diphtheria toxin, or a mutant form thereof, is subject to ultrafiltration, microfiltration, filtration and anion-exchange membrane chromatography.

In certain embodiments, the diphtheria toxin, or mutant version thereof, is CRM₁₉₇.

In certain embodiments, final liquid formulations obtained using the methods of the invention wherein ≧90% of the host cell protein and host cell impurities are removed and the yield of the diptheria toxin, or mutant form thereof, is ≧25% or ≧35%. In certain embodiments, the diptheria toxin, or mutant form thereof, is purified to a purity of ≧90%, ≧95%, or ≧98% as assessed by gel electrophoresis. Purity is calculated in reference to only the percentage of intact diphtheria toxin, or mutant form thereof (diphtheria toxin fragments are impurities). In certain embodiments, liquid formulations of the diptheria toxin, or mutant form thereof, have a concentration about ≧10 g/L or ≧50 g/L and maintain 90% purity at 2° C. for at least 6 months. In other embodiments, a 90% purity level is maintained at 25° C. for at least 5 days or at least 3 weeks as assessed by gel electrophoresis. Heterogeneity is ≦1% of the product as assessed by gel electrophoresis, endotoxin is ≦1 EU/mg as measured by a Kinetic-QCL chromogenic assay kit and aggregation is ≦0.2% or ≦0.1% by HPSEC/UV.

### BRIEF DESCRIPTION OF THE DRAWINGS

FIG. 1. SDS-PAGE results for CRM₁₉₇ purification using anion-exchange and hydroxyapatite chromatography (Lane 4 and 5) compared to an affinity-based chromatography process standard (Lane 3) and internal standard (Lane 2). A 4-12% Bis-Tris NuPAGE gel was used with 1× MES running buffer.

FIG. 2. CRM₁₉₇ stability (or integrity) at 25° C. (accelerated stability) comparing percent intact monomer of CRM₁₉₇ over time (weeks) as quantified by SDS-PAGE. The purified CRM₁₉₇ was generated by an affinity-based chromatography (squares) process, hydroxyapatite chromatography (diamonds) process and an hydroxyapatite chromatography combined with Capto-MMC™ chromatography process at lab (circles) and manufacturing scale (triangles).

FIG. 3. SDS-PAGE results for CRM₁₉₇ purification at manufacturing scale (Batch #1 and #2) compared to an internal standard. CRM₁₉₇ fragments (p37 and p25), which are difficult to remove using common purification techniques, are at low levels. A 4-12% Bis-Tris NuPAGE gel was used with 1× MES running buffer.

FIG. 4. SDS-PAGE results for CRM₁₉₇ purification comparing hydroxyapatite with Capto Adhere™ (Lane 4) and hydroxyapatite with Capto-MMC™ (Lane 5). AXP (anion-exchange product) and HAP (hydroxyapatite product) (Lane 2 and 3) are shown to display the heterogeneity clearance. A 4-12% Bis-Tris NuPAGE gel was used with 1× MES running buffer.

### DETAILED DESCRIPTION OF THE INVENTION

The present invention relates to the use of hydroxyapatite chromatography and multimodal chromatography (e.g, Capto-MMC™) for purification of diphtheria toxin and mutant forms of diphtheria toxin. The diphtheria toxin can be purified from a mixture containing the diphtheria toxin, for example, diphtheria toxin expressed in a host cell or any partially purified diphtheria toxin mixture. Conditions were determined using the hydroxyapatite chromatography such that ≧90% of the host cell protein and other residual impurities were separated from CRM₁₉₇, while maintaining a process yield of ≧50-60% prior to ultrafiltration. Conditions were determined using the hydroxaypatite and multimodal chromatography such that ≧95% of the host cell protein and other residual impurities were separated from CRM₁₉₇, while maintaining a process yield of ≧20-40%. The purification processes described herein is amenable to large scale manufacturing and has been scaled-up to accommodate 250 and 1300-L fermentation broth feeds.

The methods of the invention provide a consistent and robust purification process for diphtheria toxin, and generate intact mutant diphtheria toxin of the high quality. The mutant diphtheria toxin is able to be highly concentrated (>100 g/L) in the final bulk while maintaining protein homogeneity (e.g. intact mass and monomeric form). This is possible given the enhanced purity. As such, storage of the bulk can be done in liquid form as opposed to conventional methods which require that the bulk is lyophilized. High concentrations of diphtheria toxin are particularly important during conjugation reactions with polysaccharide as it accelerates reaction kinetics.

The resin functionalities discussed herein, e.g., hydroxyapatite and a multimodal resin, may be used in any known purification technology, such as column chromatography and membrane chromatography. Column chromatography is generally preferred due to its reusability. When the methods of the invention are used with a membrane, the absorption properties are presented on a polymeric surface. Rather than using resins packed in a column, a microporous membrane with functional groups on the internal surface area will allow for diphtheria toxin capture. These membranes could be comprised of cellulose acetate or polyvinylidene difluoride, for example, in flat sheet or hollow fiber configurations. Advantages over column chromatography include shorter operating time and less membrane volume compared to the column volume required.

As used herein, the term “diphtheria toxin” is used to refer to the naturally occurring protein. A “mutant form thereof”, when referring to diphtheria toxin, or a “mutant diphtheria toxin”, refers to sequences having ≧70%, ≧80%, ≧90%, ≧95%, ≧98%, or ≧99% identity to diphtheria toxin, and includes any known mutated forms, particularly for non-toxic mutants such as CRM₁₀₇ and CRM₁₉₇ and those described in U.S. Pat. No. 6,455,673. A “mutant form thereof” may be used with any reference to diphtheria toxin herein.

As used herein, when used with a pH or pI values, “about” refers to a variance of 0.1, 0.2, 0.3, 0.4 or 0.5 units. When used with a temperature value, “about” refers to a variance of 1, 2, 3, 4 or 5 degrees. When used with other values, such as length and weight, “about” refers to a variance of 1%, 2%, 3%, 4% 5%, or 10%.

As used herein, “clarified” refers to a sample (e.g., viable or non-viable cell) having undergone a solid-liquid separation step involving one or more of centrifugation, microfiltration, filtration or settle/decant procedure to remove host cells and/or cellular debris. A clarified fermentation broth may be fermentation supernatant. Clarification is sometimes referred to as a primary or initial recovery step and typically occurs prior to any chromatography or a similar step.

As used herein, a “mixture” comprises the protein of interest (for which purification is desired) and one or more contaminant, i.e., impurities. The mixture can be obtained directly from a cell free production system, a host cell or organism producing the polypeptide. Without intending to be limiting, examples of mixtures that can be purified according to a method of the present invention include harvested cell culture/fermentation fluid or supernatant, clarified supernatant, and conditioned supernatant. A mixture that has been “partially purified” has already been subjected to a chromatography step, e.g., non-affinity chromatography, affinity chromatography, etc. A “conditioned mixture” is a mixture, e.g., a cell culture/fermentation supernatant that has been prepared for a chromatography step, used in a method of the invention by subjecting the mixture to one or more of buffer exchange, dilution, salt addition, pH titration or filtration in order to set the pH and/or conductivity range and/or buffer matrix to achieve a desired chromatography performance. A “conditioned mixture” can be used to standardize loading conditions onto the first chromatography column. In general, a mixture can be obtained through various separation means well known in the art, e.g., by physically separating cells from other components in the broth at the end of a bioreactor run using filtration or centrifugation, or by concentration and/or diafiltration of the mixture into specific ranges of pH, conductivity and buffer species concentration.

As used herein, “polishing chromatography” refers to one or more additional chromatographic steps following capture chromatography and is used to remove residual host cell impurities and product-related impurities (diphtheria toxin heterogeneity including fragments and/or aggregated species).

Diphtheria Toxin

The sequence and the structure of the diphtheria protein have been described. See Delange et al., 1976, Proc Nat Acad Sci USA 73:69-72; and Falmagne et al., 1985, Biochim Biophys Acta 827:45-50. Mutant forms of diphtheria toxin, including but not limited to CRM₁₀₇ and CRM₁₉₇ can be purified using the methods of the invention.

Mutant forms of the toxin, such as the mutant forms described by Laird et al, 1976, J. Virology 19:220-227, and by Nicholls and Youle in Genetically Engineered Toxins, Ed: Frankel, Marcel Dekker, Inc, 1992, including CRM₁₀₇, may also be prepared by methods known in the art, for example by the methods described in Laird et al., 1976, J. Virology 19:220-227 and also by site directed mutagenesis, based on the known nucleotide sequence (Greenfield et al., 1993, Proc Nat Acad Sci 50:6953-7) of the wild type structural gene for diphtheria toxin carried by corynebacteriophage β.

The methods of the invention may also be useful for the purification of other bacterial toxins which preferably are immunologically effective antigens or carriers that have been rendered safe by chemical or genetic means for administration to a subject. Examples include inactivated bacterial toxins such as RTX-like toxins (MARTX toxins), clostridium difficile toxins, and Clostridum sp. glucosylating toxin families, tetanus toxoid, botulinum toxins, clostridial cytotoxins, pertussis toxoid, E. coli LT, E. coli ST, and exotoxin A from Pseudomonas aeruginosa. Bacterial outer membrane proteins such as, outer membrane complex c (OMPC), porins, transferrin binding proteins, pneumolysis, pneumococcal surface protein A (PspA), pneumococcal adhesin protein (PsaA), or pneumococcal surface proteins BVH-3 and BVH-11 may also be used. Other proteins, such as protective antigen (PA) of Bacillus anthracis, ovalbumin, keyhole limpet hemocyanin (KLH), human serum albumin, bovine serum albumin (BSA) and purified protein derivative of tuberculin (PPD) can also be used. The proteins are preferably proteins that are non-toxic and non-reactogenic and obtainable in sufficient amount and purity that are amenable to conjugation.

Host Cells/Cell-Free Production

Diphtheria toxin, or a mutant form thereof, can be prepared from a number of cell free production systems or host cells. For example, naturally occurring diphtheria toxin can be purified from cultures of Corynebacteria diphtheriae and other strains from a variety of publicly available sources including the American Type Culture Collection.

Cell-free production systems are well known in the art. For example, one system is based on Protein synthesis Using Recombinant Elements (PURE) (see, e.g., Shimizu et al., 2001, Nat Biotechnol 19:751-755 and Ohashi et al., 2007, Biochem Biophys Res Commun 352:270-276). Other cell-free production systems are described in Voloshin et al., 2005, Biotechnol Bioeng 91:516-21, Kim et al., 2001, Biotechnol Bioeng 74:309-16, Calhoun et al., 2005, Biotechnol Bioeng 90(5):606-13, Jewett et al., 2004, Biotechnol Bioeng 86:19-26, Jewett et al., 2004, Biotechnol Bioeng 87(4):465-72.

Diptheria toxin, and mutant forms thereof, can be expressed in C. diphtheriae or other microorganisms genetically engineered to produce the protein. Methods of genetically engineering cells to produce proteins are well known in the art. See e.g. Ausabel et al., eds. (1990), Current Protocols in Molecular Biology (Wiley, N.Y.) and U.S. Pat. Nos. 5,534,615 and 4,816,567. Such methods include introducing nucleic acids that encode and allow expression of the protein into living host cells. Other bacterial host cells include, but are not limited to E. coli cells. CRM₁₉₇ is produced by a mutant strain of Corynebacterium diphtheriae. See Uchida et al., 1973, J Biol Chem 248:3838-3844.

In certain embodiments of the invention, a mutant diphtheria toxin is produced utilizing P. fluorescens as a host expression system. See H. Jin et al., Soluble periplasmic production of human granulocyte colony-stimulating factor (G-CSF) in Pseudomonas fluorescens, Protein Expr. Purif. (2011), doi:10.1016/j.pep.2011.03.002 and U.S. Patent Application Publication No. 20090325230.

Production of Diphtheria Toxin

Diphtheria toxin, or a mutant form thereof, may be produced by methods known in the art. See, e.g., U.S. Patent Application Publication No. 20060270600. Preferably, the method involves culturing of a microorganism, e.g., a bacteria, such as C. diphtheriae. A host cell that has been engineered with nucleic acid encoding the protein of interest can be cultured under conditions well known in the art that allow expression of the protein.

CRM₁₉₇ can be produced by the methodology described by Park et al., J Exp Med (1896) 1:164-185.

In one expression system utilizing P. fluorescens, the fermentation process consists of 1st stage seed shake flask (frozen vial to flask), 2nd stage seed fermentor and production fermentor that includes a growth and induction phase. Glycerol is used as the carbon source and an isopropyl-beta-D-thiogalacto-pyranoside (IPTG) inducible promoter drives expression of the protein. The chilled cell suspension is then transferred to the recovery or purification process.

Toxin production may be monitored in a variety of known ways, such as for example, SDS PAGE, ELISA or an ADP-ribosylation assay (See Blanke et al., 1994, Biochemistry 33:5155) or by a combination of these methods.

In the production methods described herein, the pH is optimally controlled according to procedures well known to persons skilled in the art.

Pre-Processing

The mixture to be applied to hydroxyapatite and a multimodal resin, may for example be a diphtheria toxin containing supernatant, a diphtheria toxin containing cellular fraction or a diphtheria toxin containing preparation derived from a fermentation/culture supernatant, for example a concentrated supernatant, such as an ultrafiltered supernatant, or a diafiltered supernatant. The mixture to be applied is preferably processed by using methods such as flocculation, clarification and anion-exchange chromatography. In some embodiments, the mixture is processed using all three of these methods.

In the case of C. diphtheriae, where diphtheria toxin is secreted into the fermentation supernatant, the method may be carried out directly on fermentation supernatant or on a preparation derived therefrom. In the case of expression in other microorganisms, such as E. coli genetically modified to express diphtheria toxin, the diphtheria toxin may be found intracellularly, for example in the periplasm or cytoplasm. In such cases, primary recovery steps may depend upon the cellular location. The diphtheria toxin may be extracted from the cells by methods known in the art, for example as described by Skopes in Protein Purification, Principles and Practice, 3rd edn, Pub: Springer Verlag, followed by purification in accordance with the methods of the invention.

In cases where whole cells are used, the cells are preferably subjected to osmotic shock. An osmotic shock step is preferably included in the purification process following concentration of the cells. This is generally a combination of an osmolarity step change and a flocculation step. These environmental changes result in minimal cytoplasmic impurities being released and cellular debris being readily removed upon clarification. For protein molecules in the periplasmic space of bacteria, a lab scale batch osmotic shock procedure has been used to selectively release the periplasmic contents without complete cell disruption. Such a process typically begins by equilibrating fermentation broth with high molarity salt or sugar solution (soak buffer) to build high osmotic pressure within the cells. This is followed by mixing with low osmolarity buffer (shock buffer) in a batch mode for a finite period of time for release of the periplasmic contents. Release is generally followed by removal of the cell debris by a clarification method. A method for osmotically shocking cells is described in U.S. Patent Application Publication No. 20080182295.

Flocculation is a process whereby chemical agents are added to a mixture to agglomerate fine particles causing them to sediment. Many flocculants are multivalent cations such as aluminum, iron, calcium or magnesium. These positively charged molecules interact with negatively charged particles and molecules to reduce the barriers to aggregation. In addition, many of these chemicals, under appropriate pH and other conditions such as temperature and salinity, react with water to form insoluble hydroxides which, upon precipitating, link together to form long chains or meshes, physically trapping small particles into the larger aggregate. Suitable flocculants include alum, aluminium chlorohydrate, aluminium sulfate, calcium oxide, calcium hydroxide, iron(II) sulfate, iron(III) chloride, polyacrylamide, polyDADMAC, sodium aluminate, and sodium silicate. Conditions for flocculation are well known to those skilled in the art.

Additional flocculating agents are described in U.S. Pat. No. 7,326,555 as Selective Precipating Agents (SPAs). These include, but are not limited to, amine copolymers, quaternary ammonium compounds, and any respective mixtures thereof. Mixtures of these agents provide similar performance to pure forms while incorporating multiple precipitation mechanisms (i.e. primary binding sites). A mixture of these agents may be added to the mixture as the precipitation buffer or a high-cut/low-cut methodology of addition may be incorporated. More specifically, the many forms of polyethylene imine (PEI) have shown they are very effective, especially under about neutral pH conditions. Theoretically, modified PEI copolymers having relatively high molecular mass can be as efficient.

Quaternary ammonium compounds include but are not limited to the following classes and examples of commercially available products: monoalkyltrimethyl ammonium salts (examples of commercially available products include cetyltrimethylammonium bromide (CTAB) or cetyltrimethylammonium chloride, tetradecyltrimethylammonium (TTA) bromide or chloride, alkyltrimethyl ammonium chloride, alkylaryltrimethyl ammonium chloride, dodecyltrimethylammonium bromide or chloride, dodecyldimethyl-2-phenoxyethylammonium bromide, hexadecylamine chloride or bromide salt, dodecyl amine or chloride salt, and cetyldimethylethyl ammonium bromide or chloride), monoalkyldimethylbenzyl ammonium salts (examples include alkyldimethylbenzyl ammonium chlorides and benzethonium chloride (BTC)), dialkyldimethyl ammonium salts (commercial products include domiphen bromide (DB), didecyldimethyl ammonium halides, and octyldodecyldimethyl ammonium chloride or bromide), heteroaromatic ammonium salts (commercial products include cetylpyridium halides (e.g., CPC) or bromide salt and hexadecylpyridinium bromide or chloride), cis-isomer 1-[3-chloroallyl]-3,5,7-triaza-1-azoniaadamantane, alkyl-isoquinolinium bromide, and alkyldimethylnaphthylmethyl ammonium chloride (BTC 1110), polysubstituted quaternary ammonium salts (commercially available products include, but are not limited to alkyldimethylbenzyl ammonium saccharinate and alkyldimethylethylbenzyl ammonium cyclohexylsulfamate), bis-quaternary ammonium salts (product examples include 1,10-bis(2-methyl-4-aminoquinolinium chloride)-decane, 1,6-Bis(1-methyl-3-(2,2,6-trimethyl cyclohexyl)-propyldimethyl ammonium chloride}hexane or triclobisonium chloride, and the bis-quat referred to as CDQ by Buckman Brochures), and polymeric quaternary ammonium salts (includes polyionenes such as poly[oxyethylene(dimethyliminio)ethylene(dimethyliminio)ethylene dichloride], poly[N-3-dimethylammonio)propyl]N-[3-ethylneoxyethylenedimethylammonio)propyl]urea dichloride, and alpha-4-[1-tris(2-hydroxyethyle)ammonium chloride).

Clarification of the broth may be used to obtain a diphtheria toxin-containing supernatant. Bacteria may be separated from the fermentation broth by methods known in the art, such as centrifugation, settle/decant, or filtration, for example, microfiltration and diafiltration. Clarification typically involves centrifugation to pellet the solids and recover the supernatant for further processing. Alternatively, microfiltration, depth filtration, or a pH based precipitation (acid or base) may be used to filter away the solids, with the filtrate recovered for further purification. Clarification can also involve a combination of these steps, for example, centrifugation coupled with microfiltration or depth filtration. See, e.g., Wang et al., 2006, Biotechnol Bioeng 94:91-104.

Filtration to clarify the broth, which is optionally flocculated, may be effected by methods known in the art, for example with membranes such as hollow fibre or spiral wound membranes, such as by means of a 0.1 or 0.2 μm filter, for example a hollow fiber filter, such as that obtainable from A/G Technology, or a 0.4 or 0.65 μm hollow fibre or spiral wound membrane, or a 300K or 500K filter.

Diafiltration may be used to reduce ionic strength by removing salts and other ions smaller than the molecular weight cut off size of the diafiltration membrane. The reduction in ionic strength has benefits for the ensuing ion exchange step, since at reduced ionic strength, less toxin is retained by the ion exchange matrix and yield is thus improved. Furthermore, a partial purification is achieved by the diafiltration membrane. Diafiltration may thus be carried out against a low ionic strength buffer, for example Tris, Tricine, MES, Bis-Tris, TES, MOPS, inorganic salts (e.g., sulfates and phosphates), in a concentration of from about 0.1 mM to about 100 mM, preferably from about 10 to about 50 mM, for example 10 mM, having a pH of from about 6.5 to about 9.0, preferably from about 6.9 to about 8.0 for example about 7.4 to 7.6, using for example a 30,000 dalton nominal molecular weight cut off membrane which will result in removal of salts, low molecular weight media components and secreted proteins less than 30 000 dalton molecular weight. Such components may otherwise bind to the ion exchange matrix and reduce its capacity to bind toxin.

The combination of diafiltration and ultrafiltration not only reduces the ionic strength, but serves as an initial purification and allows the volume to be reduced. This means that smaller columns may be used, thereby reducing the time required for the chromatographic steps to be carried out.

For ease of handling, particularly where large volumes are concerned, such as would be the case for industrial scale purification for pharmaceutical purposes, a degree of concentration of the supernatant may be effected prior to the ion exchange step. The cell free supernatant may be concentrated, generally 5 to 50 fold, preferably 15 to 25 fold, such as 20 fold, using protein concentration methods known in the art, for example by means of ultrafiltration with porous materials for example in the form of filters, membranes or hollow fibres. For ease of handling, filters are preferred. For ultrafiltration/concentration, filters having a molecular weight cut off smaller, preferably 20% smaller than diphtheria toxin, are preferred, preferably 30 KDa filters (i.e. filters which have a 30,000 dalton nominal molecular weight). Suitable materials for such filters are known in the art and include polymeric materials such as mixed cellulose, polyether sulfone or PVDF, for example polysaccharides such as cellulose, and polysulfones. Preferred materials are those which have a lower capacity or ability to absorb diphtheria toxin. Cellulose filters are particularly preferred, for example filters made from regenerated cellulose such as the YM based filters and other membranes which have little protein binding capacity for example the Flat plate tangential flow bioconcentrators produced by Amicon.

Anion-exchange chromatography may be employed singularly or in a combination of anion-exchange substituents. In this regard various anionic substituents may be attached to matrices in order to form anionic supports for chromatography. Anionic exchange substituents include diethylaminoethyl (DEAE), trimethylaminoethyl acrylamide (TMAE), quaternary aminoethyl (QAE) and quaternary amine (Q) groups. Cellulosic ion exchange resins such as DE23, DE32, DE52, CM-23, CM-32 and CM-52 are available from Whatman Ltd. Maidstone, Kent, U.K. Sephadex-based and cross-linked ion exchangers are also known. For example, DEAE- and QAE-Sephadex, and DEAE- and Q-Sepharose are all available from GE Healthcare, Piscataway, N.J.

A conventional ion exchange resin may be used. Examples include Q sepharose and diethylaminoethyl (DEAE) and quaternary amine resins. The anion-exchange material may be packed into a column, whose size will be dependent upon the volume of culture supernatant to be used. The appropriate column size may be determined by those skilled in the art according to the total protein in the concentrated media. Generally, for large scale fermentation of the order of 40-50 liters may be applied to columns of volume about 1 L. The column may first be equilibrated with a buffer for example a low ionic strength buffer such as HEPES, Tris, Tricine, MES, Bis-Tris, TES, MOPS, inorganic salts (e.g., sulfates or phosphates), in a concentration of from about 0.1 mM to about 100 mM, preferably from about 10 to about 50 mM, for example 10 mM, having a pH of from about 5 to about 8, preferably from about 6.5 to about 9.0 for example about 6.9 to 7.6, for example the buffer used to diafilter the concentrated supernatant, such as 10 mM Tris, 20 mM KCl, pH 7.0. The fermentation supernatant or concentrate may then be loaded, the column washed with a buffer of low ionic strength and the same pH as the equilibration buffer to wash off any unbound protein, for example the equilibration buffer.

The equilibration or wash salts used can, for example, be chosen among inorganic salts for ion-exchange chromatography, such as lithium chloride, sodium chloride, potassium chloride, magnesium chloride, calcium chloride, barium chloride, sodium acetate, lithium perchlorate, sodium sulfate, magnesium sulfate, potassium phosphate and potassium sulfate, or other elution salts known to the person of skill in the art.

The mixture may be applied to an anion-exchange chromatography column, under conditions that are such that the diphtheria toxin is immobilized to the column. Any anion-exchange material may be used, but use is advantageously made of strong or weak anion-exchange chromatography columns that are commercially available, such as for example columns with functional groups selected from the group consisting of aminoethyl, diethylaminoethyl, dimetylaminoethyl, polyethyleneimine, trimethylaminomethyl, trimethylaminohydroxypropyl, diethyl-(2-hydroxypropyl)aminoethyl, quaternized polyethyleneimine, triethylaminoethyl, trimethylaminoethyl and 3-trimethylamino-2-hydroxypropyl. Among these, strong anion-exchangers with functional groups comprising quaternary amine are preferred, e.g., trimethylaminomethyl, trimethylaminohydroxypropyl, diethyl-(2-hydroxypropyl)aminoethyl, quaternized polyethyleneimine, triethylaminoethyl, trimethylaminoethyl and 3-trimethylamino-2-hydroxypropyl. The conditions under which the diphtheria toxin is immobilized to the column may be easily ascertained by the skilled person without undue experimentation.

Elution of the immobilized toxin is generally performed using a concentration gradient of a solution of an elution salt, in accordance with known chromatographic procedure. The elution salt used can, for example, be chosen among inorganic elution salts for ion-exchange chromatography, such as lithium chloride, sodium chloride, potassium chloride, magnesium chloride, calcium chloride, barium chloride, sodium acetate, lithium perchlorate, sodium sulfate, magnesium sulfate, potassium phosphate and potassium sulfate, or other elution salts known to the person of skill in the art.

Bound toxin may then be eluted in a variety of ways. These include altering the pH or increasing the ionic strength of the buffer. Thus toxin may be eluted by a gradient increase of buffer with high ionic strength, such as HEPES, Iris, Tricine, MES, Bis-Tris, TES, MOPS or phosphate, in a concentration of from about 10 mM to about 1.0 M, preferably from about 10 mM to about 500 mM, containing salts like NaCl, KCl, or ammonium sulphate at a concentration of from about 0.1 M to 1.0 M. These buffers may have a pH of from about 6.5 to about 8.5, preferably from about 6.5 to about 8 for example about 6.9 to 7.6. One example of a preferred buffer is 10 mM Tris, 1 mM potassium phosphate containing 100 mM KCl at pH 7.0. The protein will be eluted between about 60 to 90 mM KCl in the buffer.

In certain embodiments, the mixture is subject to one pass on anion-exchange chromatography. In other embodiments, the mixture is subject to more than one pass, e.g., 2, 3, or 4 passes, on anion-exchange chromatography. Where more than on pass on anion-exchange chromatography is performed, the passes may be on the same column or membrane or different columns or membranes.

Column Chromatography

The present invention provides a method of purifying diphtheria toxin, or a mutant form thereof, from a culture of diphtheria toxin (or a mutant version thereof) producing bacteria said method comprising a hydroxapatite chromatography step and a multimodal chromatography step. Either step may be performed first followed by the other step.

The chromatographic steps may be carried out using matrices as appropriate in batch or column form, the latter being preferred for both speed and convenience. The matrix may be a conventional support as known in the art for example inert supports based on cellulose, polystyrene, acrylamide, silica, fluorocarbons, cross-linked dextran or cross-linked agarose.

Hydroxyapatite

Hydroxyapatite chromatography is a method of purifying proteins that utilizes an insoluble hydroxylated calcium phosphate [Ca₁₀(PO₄)₆(OH)₂ or Ca₅(PO₄)₃OH)₂] which forms both the matrix and ligand. Functional groups consist of pairs of positively charged calcium ions (C-sites) and clusters of negatively charged phosphate groups (P-sites). The interactions between hydroxyapatite and proteins are complex and mixed-mode. In one method of interaction, however, positively charged amino groups on proteins associate with the negatively charged P-sites and protein carboxyl groups interact by coordination complexation to C-sites. See Shepard, 2000, J. of Chromatography 891:93-98.

A number of chromatographic supports may be employed in the preparation of HA columns, the most extensively used are Type I and Type II hydroxyapatite. Type I has a high protein binding capacity and better capacity for acidic proteins. Type II, however, has a lower protein binding capacity, but has better resolution of nucleic acids and certain proteins. The choice of a particular hydroxyapatite type can be determined by the skilled artisan.

Various hydroxyapatite chromatographic resins are available commercially, and any available form of the material can be used in the practice of this invention. In one embodiment of the invention, the hydroxyapatite is in a crystalline form. Hydroxyapatites for use in this invention may be those that are agglomerated to form particles and sintered at high temperatures into a stable porous ceramic mass.

The particle size of the hydroxyapatite may vary widely, but a typical particle size ranges from 1 μm to 1,000 μm in diameter, and may be from 10 μm to 100 μm. In one embodiment of the invention, the particle size is 20 μm. In another embodiment of the invention, the particle size is 40 μm. In yet another embodiment of the invention, the particle size is 80 μm.

This invention may be used with hydroxyapatite resin that is loose or packed in a column. In one embodiment of the invention, ceramic hydroxyapatite resin is packed in a column. The choice of column dimensions can be determined by the skilled artisan. In one embodiment of the invention, a column diameter of at least 0.5 cm with a bed height of about 20 cm may be used for small scale purification. In an additional embodiment of the invention, a column diameter of from about 35 cm to about 60 cm may be used. In yet another embodiment of the invention, a column diameter of from 60 cm to 85 cm may be used.

Buffer Compositions and Loading Conditions

Before contacting the hydroxyapatite resin with the mixture, it may be necessary to adjust parameters such as pH, ionic strength, and temperature and in some instances the addition of substances of different kinds. Thus, it can be necessary to perform an equilibration of the hydroxyapatite matrix by washing it with a solution (e.g., a buffer for adjusting pH, ionic strength, etc., or for the introduction of a detergent) bringing the necessary characteristics for purification of the diphtheria toxin mixture.

In combination binding/flow-through mode hydroxyapatite chromatography, the hydroxyapatite matrix is equilibrated and washed with a solution, thereby bringing the necessary characteristics for purification of the diphtheria toxin preparation. In one embodiment of the invention, the matrix may be equilibrated using a solution containing from 0.01 to 2.0 M potassium chloride or sodium chloride at slightly basic to slightly acidic pH. The equilibration buffer may also contain 0 to 20 mM potassium phosphate or sodium phosphate, in another embodiment it may contain 1 to 10 mM potassium phosphate, in another embodiment it may contain 2 to 5 mM potassium phosphate, in another embodiment it may contain 0 mM potassium phosphate, and in another embodiment may contain 3 mM potassium phosphate. The equilibration buffer may contain 0.01 to 2.0 M potassium chloride or sodium chloride, in one embodiment, 0.025 to 0.5 M potassium chloride or sodium chloride, in another embodiment, 0.05 M potassium chloride or sodium chloride, and in another embodiment, 0.1 M potassium chloride or sodium chloride. The pH of the load buffer may range from 6.5 to 8.0. In one embodiment, the pH may be from 6.8 to 7.6, and in another embodiment the pH may be 7.0. The equilibration buffer may contain other components including but not limited to CaCl₂, MgCl₂, sulfate, acetate, glycine, arginine, imidazole, succinate, and CaEDTA PO₄. Specifically in one embodiment 0 to 10 mM calcium chloride, in another embodiment it may contain 0 mM CaCl₂, and in another embodiment it may contain 1 mM CaCl₂. The equilibration buffer may contain 5 to 200 mM MOPS, in another embodiment it may contain 20 mM MOPS, and in another embodiment it may contain 50 mM MOPS.

The diphtheria toxin mixture may also be buffer exchanged or diluted into an appropriate buffer or load buffer in preparation for binding/flow-through mode hydroxyapatite chromatography. In one embodiment of the invention, the diphtheria toxin preparation may be buffer exchanged into a load buffer containing 0.01 to 2.5 M potassium chloride or sodium chloride at slightly acidic to slightly basic pH. The load buffer may further contain 1 to 10 mM potassium phosphate or sodium phosphate, in another embodiment it may contain 2 to 8 mM potassium phosphate or sodium phosphate, in another embodiment it may contain 3 to 7 mM potassium phosphate or sodium phosphate, and in another embodiment may contain 5 mM potassium phosphate or sodium phosphate. The load buffer may contain 0.2 to 2.5 M NaCl in one embodiment, 0.2 to 1.5 M NaCl, in another embodiment, 0.3 to 1.0 M NaCl, and in another embodiment, 110 mM NaCl. The pH of the load buffer may range from 6.5 to 8.0. In one embodiment, the pH may be from 6.5 to 7.6, and in another embodiment the pH may be 7.1.

The contacting of a toxin mixture to the hydroxyapatite resin in either binding mode, flow-through mode, or combinations thereof may be performed in a packed bed column, a fluidized/expanded bed column containing the solid phase matrix, and/or in a simple batch operation where the solid phase matrix is mixed with the solution for a certain time.

After contacting the hydroxyapatite resin with the toxin mixture there is a performed washing procedure. The washing buffers employed will depend on the nature of the hydroxyapatite resin, the mode of hydroxyapatite chromatography being employed, the resin may be washed using a solution containing from 0.01 to 1 M potassium chloride or sodium chloride at slightly basic to slightly acidic pH. For example, the wash buffer may contain 0.1 to 20 mM potassium phosphate or sodium phosphate, in another embodiment it may contain 0.1 to 10 mM potassium phosphate or sodium phosphate, in another embodiment it may contain 0.1 to 5 mM potassium phosphate or sodium phosphate, in another embodiment it may contain 0.5 mM potassium phosphate or sodium phosphate, and in another embodiment may contain 3 mM potassium phosphate or sodium phosphate. The wash buffer may contain 0 to 1 M potassium chloride or sodium chloride, in one embodiment, 0.025 to 0.5 M potassium chloride or sodium chloride, in another embodiment, 0.4 M potassium chloride or sodium chloride, and in another embodiment, 0.06 M potassium chloride or sodium chloride. The pH of the wash buffer may range from 6.5 to 8.0. In one embodiment, the pH may be from 6.8 to 7.6, and in another embodiment the pH may be 7.2, in another embodiment, 7.4. The wash buffer may contain other components including but not limited to CaCl₂, MgCl₂, sulfate, acetate, glycine, arginine, imidazole, succinate, or CaEDTA PO₄.

Specifically in one embodiment 0 to 10 mM calcium chloride, in another embodiment it may contain 0 mM CaCl₂, and in another embodiment it may contain 1 mM CaCl₂. The wash buffer may contain 5 to 200 mM MOPS, in another embodiment it may contain 20 mM MOPS, and in another embodiment it may contain 50 mM MOPS. Wash buffer can be applied in step or gradient mode.

In binding mode, the toxin may be eluted from the column after a single or multiple washing procedures. Elution occurs by i) step elution with an elution buffer; ii) gradient elution with an elution buffer; iii) both step and gradient with elution buffers; or iv) multiple gradients and step elutions with elution buffers. In one embodiment, for elution of the diphtheria toxin from the column, this invention uses a high ionic strength phosphate buffer containing 0 to 1 M potassium chloride or sodium chloride at slightly basic to slightly acidic pH. The elution buffer may further contain 20 to 100 mM potassium phosphate or sodium phosphate, in another embodiment it may contain 20 to 80 mM potassium phosphate or sodium phosphate, in another embodiment it may contain 30 to 60 mM potassium phosphate or sodium phosphate, in another embodiment it may contain 40 mM potassium phosphate or sodium phosphate, and in another embodiment may contain 50 mM potassium phosphate or sodium phosphate. The elution buffer may contain 0 to 1 M potassium chloride or sodium chloride, in one embodiment, 0.025 to 0.5 M potassium chloride or sodium chloride, in another embodiment, 0.5 M potassium chloride or sodium chloride, and in another embodiment, 0.06 M potassium chloride or sodium chloride. The pH of the elution buffer may range from 6.5 to 8.0. In one embodiment, the pH may be from 6.8 to 7.6, and in another embodiment the pH may be 7.2, in another embodiment, 7.0. The elution buffer may contain other components including but not limited to CaCl₂, MgCl₂, acetate, glycine, arginine, imidazole, succinate, or CaEDTA PO₄. Specifically in one embodiment 0 to 1 M magnesium chloride, calcium chloride, sodium sulfate, or ammonium sulfate, in another embodiment it may contain 0 mM CaCl₂, and in another embodiment it may contain 1 mM CaCl₂, and in another embodiment it may contain 1 M MgCl₂. The elution buffer may contain 5 to 200 mM MOPS, in another embodiment it may contain 20 mM MOPS, and in another embodiment it may contain 50 mM MOPS. The elution buffer may be altered for elution of the toxin from the column in a continuous or stepwise gradient.

In flow-through mode and combination binding/flow-through mode, the purified diphtheria toxin obtained after a wash of the column may be pooled with other purified diphtheria toxin fractions.

In certain embodiments, elution occurs by i) step elution with an elution buffer comprising ≧about 30 mM potassium chloride or sodium chloride or about ≧15 mM potassium phosphate or sodium phosphate; ii) gradient elution comprising from about 10 to about 25 mM potassium phosphate or sodium phosphate or from about 100 mM to 2 M potassium chloride or sodium chloride; or iii) a pH change of ≧0.3 pH units.

After use, the hydroxyapatite column may optionally be cleaned, sanitized, and stored in an appropriate agent, and optionally, re-used.

The hydroxyapatite used in the invention may be in one of a number of forms known in the art. The hydroxyapatite may be in the form of crystals, a gel or a resin. The normal crystalline form may alternatively be sintered at high temperatures to modify it to a ceramic form (Bio-Rad). Preferably the hydroxyapatite is in the form of a gel. Preferably, the gel is packed into a column, as commonly used in chromatography purification.

If the hydroxyapatite is in particulate form, preferably the particles have a diameter of 20 μM or more, preferably 40 μM or more, preferably 80 μM or more.

Crystalline hydroxyapatite was the first type of hydroxyapatite used in chromatography, but it was limited by structural difficulties. Ceramic hydroxyapatite (cHA) chromatography was developed to overcome some of the difficulties associated with crystalline hydroxyapatite, such as limited flow rates. Ceramic hydroxyapatite has high durability, good protein binding capacity, and can be used at higher flow rates and pressures than crystalline hydroxyapatite. See Vola et al., BioTechniques 14:650-655 (1993).

Hydroxyapatite has been used in the chromatographic separation of proteins, nucleic acids, as well as antibodies. In hydroxyapatite chromatography, the column is normally equilibrated, and the sample applied, in a low concentration of phosphate buffer and the adsorbed proteins are then eluted in a concentration gradient of phosphate buffer. See Giovannini, 2000, Biotechnology and Bioengineering 73:522-529. Sometimes shallow gradients of sodium phosphate are successfully used to elute proteins, while in other instances concentration gradients up to 400 mM sodium phosphate have been used with success. See, e.g., Stanker, 1985, J. Immunological Methods 76:157-169 (10 mM to 30 mM sodium phosphate elution gradient); Shepard, 2000, J. Chromatography 891:93-98 (10 mM to 74 mM sodium phosphate elution gradient); Tarditi, 1992, J. Chromatography 599:13-20 (10 mM to 350 mM sodium phosphate elution gradient).

Multimodal Chromatography Supports

Multimodal chromatography involves the use of solid phase chromatographic supports that employ multiple chemical mechanisms to adsorb proteins or other solutes. Mixed mode chromatography is sometimes also used to describe such chromatography, and such terms are used interchangeably herein. Examples of multimodal chromatographic supports include but are not limited to chromatographic supports that exploit combinations of two or more of the following mechanisms: anion-exchange, cation exchange, hydrophobic interaction, hydrophilic interaction, hydrogen bonding, pi-pi bonding, and metal affinity. Two such multimodal ion exchange adsorbents are commercially available from GE Healthcare, the Capto Adhere™ and Capto-MMC™ media. These combine strong anion (e.g., amino group) and weak cation exchange groups, respectively, with hydrophobic aromatic groups.

Multimodal chromatography supports provide unique selectivities that cannot be reproduced by single mode chromatography methods such as ion exchange. Multimodal chromatography provides potential cost savings, longer column lifetimes and operation flexibility compared to affinity based methods. However, the development of multimodal chromatography protocols can place a heavy burden on process development since multi-parameter screening is required to achieve their full potential. Method development is complicated, unpredictable, and may require extensive resources to achieve adequate recovery due to the complexity of the chromatographic mechanism.

Multimodal chromatography refers to chromatography that substantially involves a combination of two or more chemical mechanisms. In some embodiments, the combination results in unique selectivities that cannot be achieved by a single mode support. In certain embodiments, the multimodal resin comprises a negatively charged part and a hydrophobic part. In one embodiment, the negatively charged part is an anionic carboxylate group or anionic sulfo group for cation exchange. Examples of such supports include, but are not limited to, Capto-MMC™ (GE Healthcare). See Table 1.

Various other multimodal chromatography media are available commercially. While multimodal resins that do not comprise a negatively charged part and a hydrophobic part are not expected to behave similarly to Capto-MMC™, optimal conditions can be determined for other multimodal resins using the methods described herein. Commercially available examples include but are not limited to ceramic hydroxyapatite (CHT) or ceramic fluorapatite (CFT), MEP-Hypercel™, Capto-Adhere™, Bakerbond™ Carboxy-Sulfon™ and Bakerbond™ ABx™ (Advantor Performance Materials Inc., Phillipsburg, N.J.). See Table 1.

TABLE 1
Summary of Multimodal Chromatography Media

| Adsorbent | Proposed | Base | |
|---|---|---|---|
| Name | Mechanism | Matrix | Ligand Chemistry |
| GE Healthcare Capto-MMC ™ | Weak cation- exchange and hydrophobic interaction | Agarose | 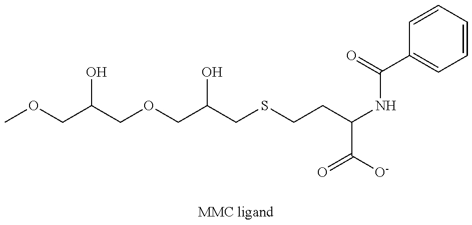 [SMILES: COCC(O)COCC(O)CSCCC(NC(=O)c1ccccc1)C(=O)[O-]] |
| GE Healthcare Capto Adhere | Strong anion- exchange and hydrophobic interaction | Agarose | 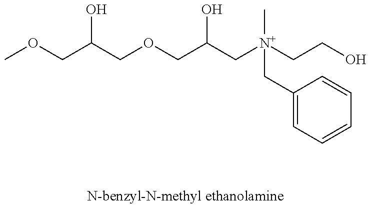 [SMILES: COCC(O)COCC(O)C[N+](C)(CCO)Cc1ccccc1] |
| Pall MEP Hypercel | Hydrophobic charge induction (pKa = 4.8, antibody selective ligand) | Cellulose | 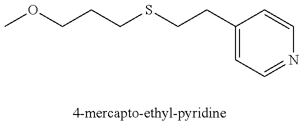 |
| Pall HEA HyperCel | Hydrophobic charge induction (pKa = 8.0, aliphatic substituent) | Cellulose | 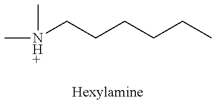 |
| Pall PPA HyperCel | Hydrophobic charge induction (pKa = 8.0, aromatic substituent) | Cellulose | 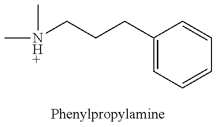 |
| Baker Bond | Strong and weak | Silica | —COOH and —S groups |
| ABx | cation exchange | | |
| Baker Bond | Weak cation and | Polymeric | —COOH and polyethylene imine (—C₂H₅N) |
| ABx | weak anion- | resin | |
| | exchange | | |
| Bio-Rad | Cation-exchange | Hydroxy- | Ca₁₀(PO₄)₆(OH)₂ |
| Ceramic | and coordination | apatite | |
| Hydroxyapatite | bonds (between | | |
| | Ca²⁺ and | | |
| | carboxyl/ | | |
| | phosphoryl | | |
| | groups) | | |

The chromatograph support may be practiced in a packed bed column, a fluidized/expanded bed column, and/or a batch operation where the multimodal support is mixed with the diphtheria toxin mixture for a certain time. A solid phase chromatography support can be a porous particle, nonporous particle, membrane, or monolith. The term “solid phase” is used to mean any non-aqueous matrix to which one or more ligands can adhere or alternatively, in the case of size exclusion chromatography, it can refer to the gel structure of a resin. The solid phase can be any matrix capable of adhering ligands in this manner, e.g., a purification column, a discontinuous phase of discrete particles, a membrane, filter, gel, etc. Examples of materials that can be used to form the solid phase include polysaccharides (such as agarose and cellulose) and other mechanically stable matrices such as silica (e.g. controlled pore glass), poly(styrenedivinyl)benzene, polyacrylamide, ceramic particles and derivatives of any of these.

In some embodiments, the multimodal support is packed in a column of at least 5 mm internal diameter and a height of at least 25 mm, such as those incorporated into liquid handling robotics. Such embodiments are useful, e.g., for evaluating the effects of various conditions.

Another embodiment employs the multimodal support, packed in a column of any dimension required to support preparative applications. Column diameter may range from less than 1 cm to more than 1 meter, and column height may range from less than 1 cm to more than 50 cm depending on the requirements of a particular application. Commercial scale applications will typically have a column diameter (ID) of 20 cm or more and a height of at least 25 cm.

Appropriate column dimensions can be determined by the skilled artisan.

In some embodiments, the multimodal resin is in a column. The column may be low-pressure (≦3 bar of back pressure). In some embodiments, the column is packed with a medium having a particle diameter of about >30 μm, for example, about 30 to about 100 μm. In some embodiments, the column has a pore size of about 100 to about 4000 angstroms, for example, about 150 to about 300 angstroms. In some embodiments, the column length is about 10 to about 50 cm, for example, about 25 to about 35 cm. Preferably, the column is a preparative column, meaning preparative scale and/or preparative load. The preparative-scale column typically has a diameter of at least about 1 cm, for example, at least about 6 cm, up to and including about 15 cm, about 60 cm, or higher. The medium of the column may be any suitable material, including polymeric-based media, silica-based media, or methacrylate media. In one embodiment, the medium is agarose-based.

The column may be an analytical or preparative column. The amount of diphtheria toxin loaded onto the column is generally about 0.01 to about 40 g diphtheria toxin/liter bed volume, for example, about 0.02 to about 30 g diphtheria toxin/liter bed volume, about 1 to about 15 g diphtheria toxin/liter bed volume, or about 3 to about 10 g diphtheria toxin/liter bed volume. The preparative-load column has a load of molecule of at least about 0.1 g diphtheria toxin/liter bed volume, for example, at least about 1 g diphtheria toxin/liter.

The flow rate is generally about 50 to about 600 cm/hour, or about 4 to about 20 column volumes (CV)/hour, depending on the column geometry. Appropriate flow velocity can be determined by the skilled artisan.

The temperature may be in the range of about 2 to about 30° C., such as room temperature.

In preparation for contacting the diphtheria toxin preparation with the multimodal support, in some embodiments, the chemical environment inside the column is equilibrated. This is commonly accomplished by flowing an equilibration buffer that is equivalent or similar to the loading buffer conditions, such as a Tris, MES, MOPS, HEPES, potassium phosphate or sodium phosphate buffer solution with or without inorganic salts, for example sodium chloride or potassium chloride, through the column to establish the appropriate pH; conductivity; and other pertinent variables. The equilibration buffer is isotonic with a conductivity ≦30 mS/cm and commonly has a pH in the range from about 6.5 to about 8. The equilibration buffer could alternatively have a conductivity ≧30 mS/cm and commonly has a pH in the range from about 6.2 to 7.8 due to its multimodal structure.

In one embodiment of the invention, the matrix may be equilibrated using a solution containing from 0.01 to 0.5 M potassium chloride or sodium chloride at slightly basic to slightly acidic pH. The equilibration buffer may also contain 0 to 100 mM potassium phosphate or sodium phosphate, in another embodiment it may contain 10 to 25 mM potassium phosphate or sodium phosphate, in another embodiment it may contain 10 mM potassium phosphate or sodium phosphate, in another embodiment it may contain 20 mM potassium phosphate or sodium phosphate, and in another embodiment may contain 25 mM potassium phosphate or sodium phosphate. The equilibration buffer may contain 0.01 to 0.5 M potassium chloride or sodium chloride, in one embodiment, 0.025 to 0.2 M potassium chloride or sodium chloride, in another embodiment, 0.05 M potassium chloride or sodium chloride, and in another embodiment, 0.1 M potassium chloride or sodium chloride. The pH of the load buffer may range from 6.5 to 8.0. In one embodiment, the pH may be from 6.8 to 7.3, and in another embodiment the pH may be 7.0. The equilibration buffer may contain other components, e.g., a protease inhibitor, or cocktails including but not limited to AEBSF, Leupeptin, EDTA, Aprotinin, Pepstatin, PMSF, Chymostatin, 2-Mercaptoethanol, Benzamidine, EGTA, sodium bisulfite, ethylenediaminetetracetic acid, protease inhibitor cocktails (e.g., SigmaFAST™), and lactacystin. The equilibration buffer may contain 5 to 200 mM MOPS, in another embodiment it may contain 20 mM MOPS, and in another embodiment it may contain 50 mM MOPS.

The diphtheria toxin mixture may also be buffer exchanged or diluted into an appropriate buffer or load buffer in preparation for multimodal chromatography. In one embodiment of the invention, the diphtheria toxin preparation may be buffer exchanged into a load buffer containing 0.01 to 0.5 M potassium chloride or sodium chloride at slightly acidic to slightly basic pH. The load buffer may further contain 0 to 100 mM potassium phosphate or sodium phosphate, in another embodiment it may contain 10 to 25 mM potassium phosphate or sodium phosphate, in another embodiment it may contain 10 mM potassium phosphate or sodium phosphate, and in another embodiment may contain 25 mM potassium phosphate or sodium phosphate. The load buffer may contain 0.01 to 0.5 M potassium chloride or sodium chloride in one embodiment, 0.025 to 0.2 M potassium chloride or sodium chloride, in another embodiment 0.05 M potassium chloride or sodium chloride, and in another embodiment, 0.1 M potassium chloride or sodium chloride. The pH of the load buffer may range from 6.5 to 8.0. In one embodiment, the pH may be from 6.8 to 7.3, and in another embodiment the pH may be 7.2. The load buffer may contain other components, e.g., a protease inhibitor, or cocktails including but not limited to AEBSF, Leupeptin, EDTA, Aprotinin, Pepstatin, PMSF, Chymostatin, 2-Mercaptoethanol, Benzamidine, EGTA, sodium bisulfite, ethylenediaminetetracetic acid, protease inhibitor cocktails (e.g, SigmaFAST™), and lactacystin. The load buffer may contain 5 to 200 mM MOPS, in another embodiment it may contain 20 mM MOPS, and in another embodiment it may contain 50 mM MOPS.

An elution buffer is used to elute the toxin from the mixed mode resin. Suitable elution buffers include but are not limited to HEPES, MOPS, TRIS, phosphate, BICINE, or triethanolamine containing about 0.005 to about 1 M sodium chloride, potassium phosphate, sodium sulfate, or ammonium sulfate, potassium chloride, magnesium chloride, calcium chloride, lithium sulfate, lithium chloride, sodium acetate, ammonium chloride, ethanol, urea propylene glycol, arginine, guanidine, sodium citrate, or a combination thereof buffered at pH 7 to 9. In certain embodiments, the elution buffer contains at least 0.1 M potassium chloride or sodium chloride. The diphtheria toxin may be eluted with step elution with an elution buffer having a salt concentration higher or lower than the salt concentration of elution or by gradient elution with any gradient starting with a salt concentration below or above the salt concentration of elution. In specific embodiments, elution occurs by i) step elution with an elution buffer containing ≧about 0.005 M sodium chloride, or 0 to 0.1 mM potassium chloride to about 1M sodium chloride or 1 to about 0.1 M sodium chloride or about 0.5 to about 1.0 M sodium sulfate; or ii) gradient elution from about 0.1 to about 0.5 M sodium chloride or 0.5 to about 0.1 M sodium chloride; or iii) step elution with an elution buffer at ≧pH 7.5; or iv) gradient elution from about pH 6.5 to about 9.0. Any combination or perturbation of elutions methods are applicable. The elution buffer may contain other components, e.g., a protease inhibitor cocktail, or cocktails including but not limited to AEBSF, Leupeptin, EDTA, Aprotinin, Pepstatin, PMSF, Chymostatin, 2-Mercaptoethanol, Benzamidine, EGTA, sodium bisulfite, ethylenediaminetetracetic acid, protease inhibitor cocktails (e.g., SigmaFAST™), and lactacystin. The elution buffer for step elution may contain about 0.1 to about 1.0 M NaCl, for example, 0.1±0.1 M NaCl or KCl, buffered at pH 8.5±0.1. An elution buffer for gradient elution may be any gradient that encompasses, for example, about 0.1 to about 0.5 M NaCl or 0.5 M to about 0.1 M KCl, including, but not limited to, 0 to about >1.0 M, 0.1 to about 0.5 M NaCl. The elution buffer is typically buffered at pH 7 to 9. Elution typically occurs over about 5 to about 20 column volumes.

In certain embodiments, elution is by i) step elution with an elution buffer comprising ≧about 125 mM potassium chloride or sodium chloride at pH 6.8 to 9.5; ii) gradient elution comprising from about 0.2 to about 0.3 M sodium chloride, potassium chloride, sodium sulfate, ammonium sulfate, or potassium chloride; iii) a pH change of ≧0.5 pH units within a pH range of 6.5 to 9.5; or (iv) a temperature change of ≧1° C. within a temperature of 2 to 30° C.

After use, the multimodal column may optionally be cleaned, sanitized, and stored in an appropriate agent, and optionally, re-used.

In certain embodiments, the chromatographic support is optionally washed after loading. The column can be washed to 1) remove unbound loading sample from the column prior to elution and 2) remove weakly bound impurities. For example, if loading occurs at pH 7, a wash at pH 7.5 without NaCl, may wash off some bound impurities prior to increasing salt strength for product elution. Wash strategies are often used in lieu of a gradient elution at process scale since they are simple to implement. The wash buffer typically contains Tris, HEPES, MOPS, phosphate, BICINE, or triethanolamine, having a pH between about 7.5 and about 8.0 and a conductivity <30 mS/cm when run in primary cation and secondary HIC mode.

The multimodal chromatography is preferably used as a polishing step, and thus serves for purification of a diphtheria toxin, in particular to the reduction, decrease or elimination, of host cell proteins, aggregates and product fragments.

Purified when referring to a component or fraction indicates that its relative concentration (weight of component or fraction divided by the weight of all components or fractions in the mixture) is increased by at least about 20%. As used herein, purity is calculated in reference to the intact product, i.e., diphtheria toxin fragments are considered to be impurities. In one series of embodiments, the relative concentration is increased by at least about 40%, about 50%, about 60%, about 75%, about 100%, about 150%, or about 200%. A component or fraction can also be said to be purified when the relative concentration of components from which it is purified (weight of component or fraction from which it is purified divided by the weight of all components or fractions in the mixture) is decreased by at least about 20%, about 40%, about 50%, about 60%, about 75%, about 85%, about 95%, about 98% or 100%. In still another series of embodiments, the component or fraction is purified to a relative concentration of at least about 50%, about 65%, about 75%, about 85%, about 90%, about 97%, about 98%, or about 99%.

In preferred embodiments, using the methods of the invention, ≧90%, ≧95%, or ≧98%, of the host cell protein and other protein impurities are removed and the yield of diphtheria toxin, or a mutant version thereof, is ≧30%, ≧40%, more preferably ≧50%. In certain embodiments, <0.001%, <0.0001%, <0.00001% of the host cell DNA is present.

In preferred embodiments, a diphtheria toxin, or mutant version thereof, is purified to a purity of ≧90%, ≧95%, ≧97% or ≧99% as assessed by gel SDS electrophoresis.

Electrophoresis is generally carried out under denaturing and reducing conditions, using a polyacrylamide gel such as a 4-12%, 4-20%, 10%, or 12% gel in a NuPAGE or Tris-glycine gel system such as from Invitrogen (Carlsbad, Calif.). Gels are stained overnight with a suitable stain, for example, Sypro Ruby fluorescent protein stain or Simply Blue Safe stain from Invitrogen and then imaged with a laser-induced fluorescence scanner such as a Molecular Dynamics fluoroimager 595.

The purification methods according to the invention result in a high diphtheria toxin purity (i.e. without significant levels of contaminating proteins), toxin stability, and low heterogeneity, without sacrifice to yield. Thus we have found that by carrying out a hydroxyapatite step followed by a multimodal resin, a purity of up to around 98% can be reproducibly achieved upon scale-up. In certain embodiments of the invention, the diphtheria toxin purified with the methods of the invention maintains the purity target at 2° C. and −60° C. for at least 6 months or at 25° C. for at least 5 days or at least 3 weeks, as assessed by gel electrophoresis. In certain embodiments of the invention, heterogeneity is ≦1%. In certain embodiments of the invention, the endotoxin concentration is <20 EU/mg, <10 EU/mg, or <1 EU/mg as assayed using standard methods. In certain embodiments of the invention, aggregation is <1%, <0.5%, <0.2%, or <0.1% as assayed by HPSEC/UV.

Additional Steps

The recovered diphtheria toxin obtained following hydroxyapatite and multimodal chromatography may optionally be subject to one or more of the following: ultrafiltration, microfiltration, anion-exchange membrane chromatography, and absolute filtration. In one aspect of the invention, the recovered diphtheria toxin is subject to ultrafiltration, anion-exchange chromatography, and absolute filtration.

In certain embodiments, a further purification and filtration step occurs after eluting from the multimodal resin. Such purification preferably involves one or more polishing chromatography steps (such as cation or anion-exchange chromatography, hydrophobic interaction chromatography, or hydroxyapatite chromatography) to remove residual host cell proteins, nucleic acids, and endotoxin as well as process excipients such as leached multimodal ligand. The filtration step preferably involves ultrafiltration for removal of low molecular-weight impurities and process excipients and for exchange into the final product formulation buffer. EDTA is a small molecule that is included in the purification process. A sterile filtration occurs to remove any particulates and to control bioburden (as required).

Additional Optional Steps

The eluted protein preparation may be subjected to additional purification or filtration steps either prior to, or after, the hydroxyapatite and multimodal chromatography step. Exemplary further purification steps, in addition to those described above, include dialysis; affinity chromatography; hydrophobic interaction chromatography (HIC); additional multimodal chromatography; ammonium sulphate precipitation; cation exchange chromatography; ethanol precipitation; reverse phase HPLC; chromatography on silica; chromatofocusing; and gel filtration.

In one embodiment of the invention, a complete process includes recovery and concentration of host cells followed by osmotic shock to release CRM₁₉₇ from the periplasm and subsequent flocculation of cell debris. A two-stage clarification module (centrifugation+depth filtration) then removes the cell debris. Next, anion-exchange chromatography is used to capture the CRM₁₉₇ from the protein impurities, residual media and buffer components. Further removal of endotoxin and residual proteins is achieved by a subsequent hydroxyapatite chromatography step. Ultrafiltration is used to concentrate the batch and exchange to 100 mM potassium phosphate pH 7.2. Flow-through anion-exchange membrane chromatography is used as a final polishing step, and a bioburden reduction filtration completes the process.

One process of the invention, using hydroxyapatite followed by a multimodal chromatography is shown below.

| STEP | PURPOSE |
|---|---|
| Harvest Centrifugation | Recovery and concentration of cells |
| Osmotic Shock/ | CRM197 release from cells and cell debris |
| Flocculation | flocculation |
| Clarification Centrifugation | Removal of flocculated cell debris |
| Depth Filtration | Polishing clarification of cell debris |
| Anion Exchange | Capture chromatography step, endotoxin |
| Chromatography | reduction, clearance of upstream |
| | media/buffer components |
| Hydroxyapatite | Bind-elute chromatography to increase |
| Chromatography | protein purity and decrease |
| | endotoxin, nucleic acids and other impurities |
| Capto MMC | Polishing chromatography to increase protein |
| Chromatography | purity and decrease endotoxin |
| Ultrafiltration | Exchange into formulation buffer and |
| | reduce volume |
| 0.2 μm Filter + | Residual impurity clearance |
| Membrane | |
| Chromatography | |
| 0.2 μm Final Filtration | Remove bioburden |

The diphtheria toxin, or a mutant version thereof, obtained according to the methods of the invention can be used in liquid formulations using methods well known to those skilled in the art.

The specific embodiments described herein are offered by way of example only, and the invention is to be limited only by the terms of the appended claims along with the full scope of equivalents to which such claims are entitled. Indeed various modifications of the invention, in addition to those shown and described herein will become apparent to those skilled in the art from the foregoing description and accompanying drawings. Such modifications are intended to fall within the scope of the appended claims.

### EXAMPLES

### Purification Reagents

Ammonium sulfate, calcium chloride, edentate disodium, magnesium salts, potassium phosphate monobasic, potassium chloride, potassium hydroxide, sodium chloride, sucrose, MOPS, Tris, sodium hydroxide, and hydrochloric acid were obtained from Advantor Performance Materials (Phillipsburg, N.J.). Glycerol, IPTG, polyethylenimine, and potassium phosphate dibasic were purchased from Sigma-Aldrich Co. (St. Louis, Mo.). Hydroxyapatite chromatography media was obtained from Bio-Rad Laboratories Inc. (Hercules, Calif.). Tris hydrochloride was obtained from Amresco Inc. (Solon, Ohio). Capto Q, Capto-MMC™ and Capto Adhere chromatography media was obtained from GE Healthcare (Upsala, Sweden).

### Fermentation

Approximately 200 L or 1300 L of P. fluorescens fermentation broth was prepared in a 250-L or 1500-L bioreactors using methods well-known to those skilled in the art (e.g., as disclosed by H. Jin et al., Soluble periplasmic production of human granulocyte colony-stimulating factor (G-CSF) in Pseudomonas fluorescens, Protein Expr. Purif. (2011), doi:10.1016/j.pep.2011.03.002) except scaled up. The 1300 L bulk purification process generally produces ˜9 L of purified CRM₁₉₇ at a concentration ≧50 g/L. This translates to a process recovery of ≧30% or productivities ≧0.3 g CRM₁₉₇/L fermentation.

### Example 1

### Purification using Hydroxyapatite Chromatography

The suitability of hydroxyapatite chromatography was tested. A standard anion-exchange chromatography step was included prior to hydroxyapatite chromatography to increase protein purity to ≧90% and reduce endotoxin.

A fermentation broth of 200 L was prepared as described above.

### Cell Recovery and Harvest

Recovery and concentration of the P. fluorescens cells was accomplished using continuous centrifugation. The 200 L fermentation batch was first cooled to <8° C. in the bioreactor with agitation. The bowl of the Westfalia centrifuge was brought to full speed with cooling. The fermentation broth was fed to the centrifuge to maintain a Q/Σ of about 5 E-5 L/(min m²)). The step was run to harvest the cell slurry with centrate directed to waste.

Temperature was controlled throughout the harvest step to maintain bowl (<8° C.) temperature. After each discharge, the harvested cell slurry was transferred to a tank.

### Osmotic Shock and Flocculation

In this step, the release of CRM₁₉₇ protein from the P. fluorescens periplasm was accomplished by osmotic shock and a flocculant was added to aid in clarification. Agitation was set to create vigorous mixing as resuspension buffer (50% w/v sucrose, 200 mM Tris, 100 mM EDTA pH 7.5) was added to resuspend the harvested cell slurry. Resuspended cells were then osmotically shocked by adding the resuspended batch to 4X volume of Osmotic Shock Buffer (50 mM Tris pH 7.5) with fast agitation, thereby releasing CRM₁₉₇ protein. With reduced agitation, polyethylimine (PEI) flocculant (10% w/v) was added to achieve a final concentration of 0.2% w/v PEI.

### Clarification Centrifugation and Depth Filtration

The bulk removal of cell debris was accomplished by centrifugation and depth filtration. The centrifuge was run similarly for clarification however the centrate was collected as the product and the solids were discharged as waste using centrifugation buffer (10 mM Tris, pH 7.5). Simultaneously, the collected centrate was transferred through Cuno 120ZA08A depth filters to reduce turbidity (200 L/ft² of filter area).

### Anion-Exchange

The primary capture step for CRM₁₉₇ from protein impurities, endotoxin, nucleic acids, and fermentation impurities was performed with Anion-exchange Chromatography (AEX). AEX was performed at constant linear velocity (287 cm/hr) and at room temperature using buffers at <8 ° C. The depth filter product was diluted approximately 3-fold with cold process water using in-line dilution on the chromatography skid to achieve a conductivity of 2.5 mS/cm in the diluted process stream. The diluted feed stream was then loaded directly onto the AEX column (Capto Q, GE Healthcare) that has been equilibrated with 20 mM KCl, 10 mM Tris, 0.5 mM KPi (potassium phosphate), pH 7.1. The column was washed with 10 mM Tris, 20 mM KCl, 0.5 mM KPi, pH 7.1 until baseline absorbance is obtained. The product was eluted in a step to 110 mM KCl using a 50 mM MOPS, 110 mM KCl, pH 7.0 buffer. Product collection began at 0.5 CV after the start of the step elution and concluded at 11.5 CVs.

### Hydroxyapatite Chromatography

Hydroxyapatite (HA) chromatography is a polishing step which increases protein purity and further reduces endotoxin levels. The HA process was run at a constant flow rate (10 min residence time) and executed at room temperature except for column loading which is <8° C. The HA column which was packed with CHT Ceramic HA Type I (BioRad), 40 μm resin was equilibrated with 110 mM KCl, 50 mM MOPS pH 7.0. Loading was about 9 g diphtheria toxin/L. The column was washed as described in Table 2.

TABLE 2
Hydroxyapatite chromatography wash protocols for Batch #1 and
Batch #2. Column volumes (CVs) and buffer compositions are
as described, with all buffers containing 50 mM MOPS, pH 7.0.

| Mode | Batch #1 | Batch #2 |
|---|---|---|
| Wash 1 | 10 CV 0.8M KCl | 10 CV 1M KCl |
| Wash 2 | 5 CV gradient 0.11M | 7 CV gradient 0.11M |
| | KCl | KCl |
| Wash 3 | 10 CV 0.11M KCl, 5 mM | 10 CV 0.11M KCl, 3 mM |
| | KPi | KPi |

CRM₁₉₇ was eluted from the column in a 10 CV linear gradient to 110 mM KCl, 30 mM KPi, 50 mM MOPS, pH 7.0, followed by a 5 CV hold. Product was collected in 15 CV, 20 CV or until absorbance reached baseline.

This example resulted in reproducible purifications not limited to 200-L culture/fermentation feeds wherein ≧90% of the host cell protein and host cell impurities were removed and the process yield of diptheria toxin was ≧50% by UV (Batch #1: 55% and Batch #2: 50%) prior to ultrafiltration. In certain embodiments, the diptheria toxin was purified to a purity of ≧94% as assessed by gel electrophoresis.

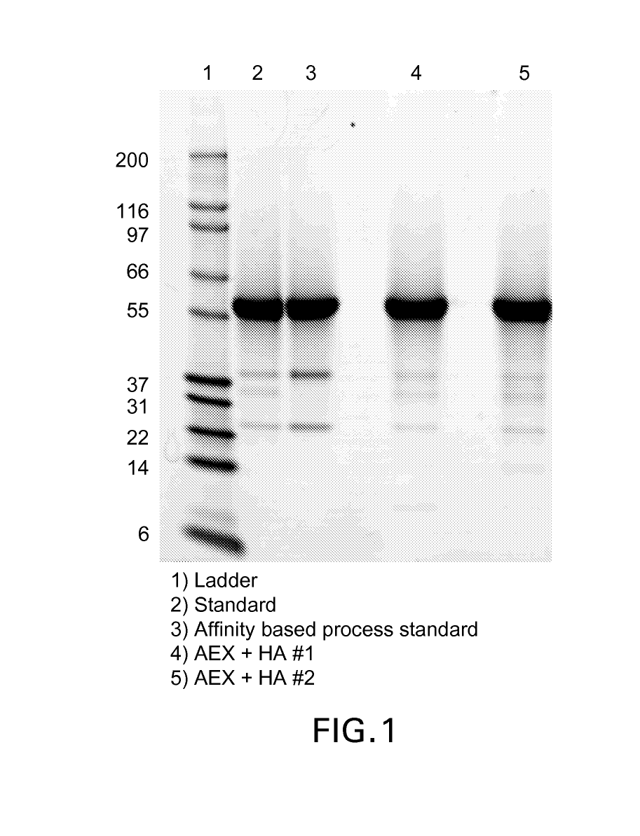

Results are shown in FIG. 1. The AEX and hydroxyapatite chromatography train produces material of similar or improved purity compared to the standard and affinity chromatography produced product (Mimetic blue resin from Prometic Life Sciences Inc. (Laval, Quebec). A 4-12% Bis-Tris NuPAGE gel was used with 1× MES running buffer. The diptheria toxin at a concentration ≧50 g/L maintained the purity target at ≧25° C. (worse case accelerated stability) for ≧5 days. Purified diphtheria toxin was typically stored at −70° C.

### Example 2

### Commercial Scale Purification using CAPTO-MMC™

### Cell Recovery and Harvest

Recovery and concentration of the P. fluorescens cells was accomplished using continuous centrifugation. The 1300 L fermentation batch is first cooled to <8° C. in the bioreactor with agitation. The bowl of the Westfalia centrifuge was brought to full speed with cooling. The fermentation broth was fed to the centrifuge to maintain a Q/Σ of about 1.25 E-4 L/(min m²)). The step was run to harvest the cell slurry with centrate directed to waste.

Temperature was controlled throughout the harvest step to maintain bowl (<8° C.) temperature. After each discharge, the harvested cell slurry was transferred to a tank.

### Osmotic Shock and Flocculation

In this step, the release of CRM₁₉₇ protein from the P. fluorescens periplasm was accomplished by osmotic shock and a flocculant was added to aid in clarification. Agitation was set to create vigorous mixing as resuspension buffer (50% w/v sucrose, 200 mM Tris, 100 mM EDTA pH 7.5) was added to resuspend the harvested cell slurry. Resuspended cells were then osmotically shocked by adding the resuspended batch to 4× volume of Osmotic Shock Buffer (50 mM Tris pH 7.5) with fast agitation, thereby releasing CRM₁₉₇ protein. With reduced agitation, polyethylimine (PEI) flocculant (10% w/v) was added to achieve a final concentration of 0.2% w/v PEI.

### Clarification Centrifugation and Depth Filtration

The bulk removal of cell debris was accomplished by centrifugation and depth filtration. The centrifuge was run similar to cell recovery for clarification however the centrate was collected as the product and the solids were discharged as waste using centrifugation buffer (10 mM Tris, pH 7.5). Simultaneously, the collected centrate was transferred through 6×1.84 m² CUNO Z16E08AA120ZA08A depth filter stacks in parallel.

### Anion-Exchange Chromatography

The primary capture step for CRM197 from protein impurities, endotoxin, nucleic acids, and fermentation impurities was performed with Anion-exchange Chromatography (AEX). AEX was performed at constant linear velocity (287 cm/hr) and at room temperature using buffers at <8 ° C. The depth filter product was diluted approximately 3-fold with cold WFI using in-line dilution on the chromatography skid to achieve a conductivity of 2.5 mS/cm in the diluted process stream. The diluted feed stream was then loaded directly onto the AEX column (Capto Q, GE Healthcare) that has been equilibrated with 20 mM KCl, 10 mM Tris, 0.5 mM KPi, pH 7.1. The column was washed with 10 mM Tris, 20 mM KCl, 0.5 mM KPi, pH 7.1 until baseline absorbance was obtained. The product was eluted in a step to 110 mM KCl using a 50 mM MOPS, 110 mM KCl, pH 7.0 buffer. Product collection began at 1 CV after the start of the step elution and concluded at 8 CVs.

### Hydroxyapatite Chromatography

Hydroxyapatite (HA) chromatography is a polishing step which increases protein purity and further reduces endotoxin levels. The HA process was run at a constant flow rate (10 min residence time) and executed at room temperature except for column loading which is <8° C. The HA column was packed with CHT Ceramic HA Type I (BioRad), 40 μm resin was equilibrated with 55 mM KCl, 50 mM MOPS pH 7.0. Cold AEX product that has been batch diluted 2-fold with <8° C., 50 mM MOPS pH 7.0 was loaded onto the column. Loading was about 10 g diphtheria toxin/L. The column was chased with equilibration buffer and washed with 8CV of 55 mM KCl, 50 mM MOPS, 3 mM potassium phosphate pH 7.2. The main product peak was eluted with an 8 CV gradient elution to 40 mM KPi, 50 mM MOPS, 55 mM KCl, pH 7.0. HA eluate was collected at the start of the elution gradient. The gradient continued for 8 CVs followed by a 6 CV of 50 mM MOPS, 55 mM KCl, 40 mM KPi, pH 7.0. Product collection ended at 10% of peak max.

### Multimodal Cation Chromatography

Multimodal cation chromatography (Capto-MMC™, GE Healthcare) is a polishing step which increases protein purity and removes aggregates. The MMC feed was first diluted 0.25-fold with 50 mM MOPS, 400 mM KCl, 25 mM KPi, 25 mM EDTA, pH 7.0. The MMC process was run at a constant flow rate (equivalent to a 10 minute residence time) and executed at room temperature. The column was equilibrated with 50 mM MOPS, 125 mM KCl, 25 mM KPi, 5 mM EDTA, pH 7.1 and the HA product loaded onto the column. Loading was about 5 g diphtheria toxin/L. The column then was washed with 5 CV of equilibration buffer and eluted via a 2.5 CV step gradient to 50 mM Tris, 200 mM KCl, 5 mM EDTA, pH 8.5 followed by a 10 CV linear gradient elution to 50 mM Tris, 500 mM KCl, 5 mM EDTA, pH 8.5. Eluate collection started immediately following initiation of the pH step gradient, and was concluded after baseline resolution had been achieved for a number of column volumes.

### Ultrafiltration

The CRM₁₉₇ protein was concentrated and diafiltered into the final formulation buffer by ultrafiltration. A 10 kDa NMWC regenerated cellulose membrane (Millipore, Billerica, Mass.) was used for the operation and performed using constant flow rates. The entire process was performed at <8 ° C. MMC product was first concentrated to about 5 to 10 g CRM197/L (based on a fixed volume) followed by a diafiltration of 20 diavolumes (DV) against Formulation Buffer (0.1M KPi, pH 7.2). The product was over-concentrated to ≧80 to 100 g CRM₁₉₇/L, followed by a retentate rinse to target a concentration of approximately ≧65 g CRM₁₉₇/L for the diafiltration product (DFP).

### Prefiltration and Membrane Chromatography

Turbidity reduction was conducted by the prefilter (0.5/0.2 μm PES membrane, Millipore Corp.) and additional endotoxin clearance was provided by membrane chromatography (Q membrane, Sartorius), in flow-through mode. The concentrated ultrafiltration material was allowed to equilibrate at room temp prior to performing the prefiltration and membrane chromatography. The concentrated product was filtered through a 0.5/0.2 μm PES membrane capsule and Sartobind Q membranes that have been pre-sanitized (0.5 N NaOH) and equilibrated with Formulation Buffer. A recovery flush of Formulation Buffer is passed through the filter/membrane setup to target a final concentration of approximately 60g/L for the Membrane Chromatography Product (MCP).

### Bioburden Reduction or Sterile Filtration

The MCP was passed through a second 0.5/0.2 μm PES membrane capsule prior to dispensing. Bulk was dispensed aseptically and frozen at −70° C.

This process resulted in reproducible purifications not limited to 1 L, 15 L, 30 L, 250 L, and 1300 L culture/fermentation feeds wherein ≧95% of the host cell protein and host cell impurities were removed and the yield of diptheria toxin was ≧30% by UV. In certain embodiments, the diptheria toxin was purified to a purity calculated in reference to the intact product of ≧98% as assessed by gel electrophoresis. The diptheria toxin was at a concentration about ≧50 g/L or ≧60 g/L and maintained the purity target at <8° C. and <−60° C. for at least ≧6 months and at ≧25° C. (worse case accelerated stability) for ≧3 weeks as assessed by gel electrophoresis.

Mean endotoxin at the manufacturing scale was ≦1 EU/mg as measured by a Kinetic-QCL chromogenic assay kit and aggregation ≦0.1% by HPSEC (analytical high performance size exclusion chromatography)/UV. Heterogeneity was not detected or ≦1%, an example being the undetectable p37 and p25 CRM197 fragments, hence removal by the chromatography resins.

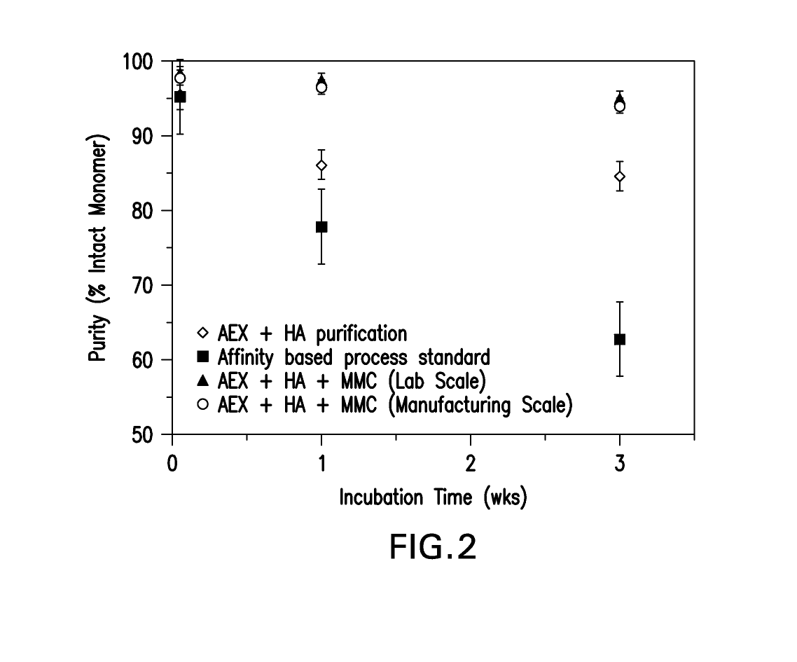

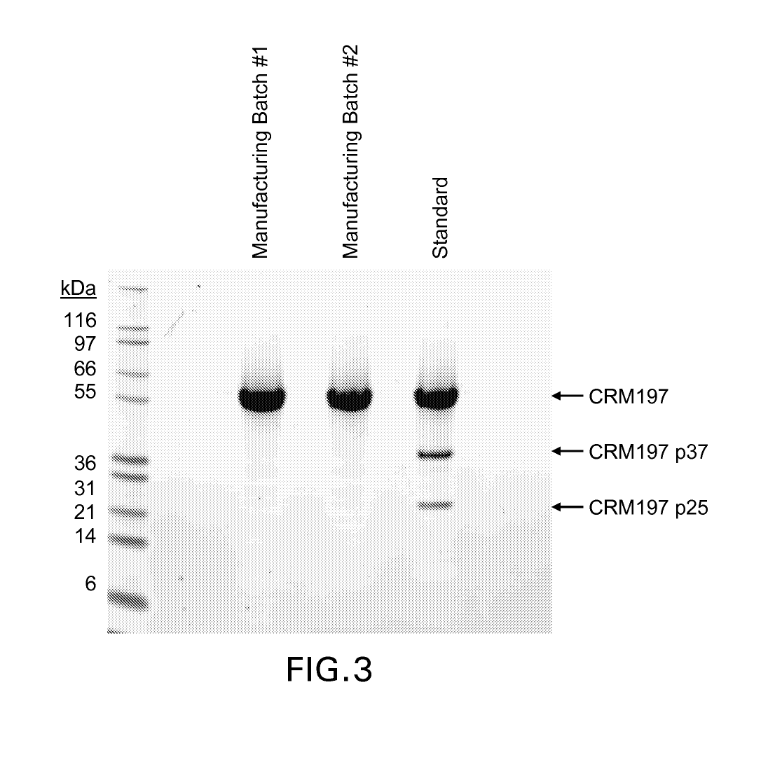

Results are shown in Table 3, FIG. 2 and FIG. 3. The AEX, hydroxyapatite, and Capto-MMC™ chromatography train produced diphtheria toxin of improved purity (percent intact monomer and host cell impurities), homogeneity, and stability compared to the AEX and hydroxyapatite and affinity chromatography produced product (Mimetic blue resin from Prometic Life Sciences (Laval, Quebec). A 4-12% Bis-Tris NuPAGE gel was used with 1× MES running buffer. Data points and error bars represent the average and standard deviation for 3-5 replicates.

TABLE 3
Hydroxyapatite and Multimodal process attributes
for 1300 L fermentation feeds

| | | Manufacturing |
|---|---|---|
| Attribute | Manufacturing Batch #1 | Batch #2 |
| Purity or Intact | 99 | 98 |
| Diphtheria Toxin | | |
| Endotoxin | <1 EU/mg | <1 EU/mg |
| DNA | 1.4E−05% | 1.3E−05% |
| Aggregation | <0.1% | <0.1% |
| Heterogeneity | None detected or <1% | None detected or <1% |
| Stability (purity >95%) | >3 weeks at 25° C. | >3 weeks at 25° C. |
| Process Recovery | 40% | 37% |

### Example 3

### Comparison of Multimodal Resins

A batch of hydroxyapatite product was divided and run on both Capto Adhere and Capto-MMC™ to help evaluate between the two resins.

The process steps for the Capto Adhere process are summarized in Table 4. A 370 mL column with a residence time of 8 minutes was used. Loading was ˜8 mg diphtheria toxin/mL for this batch. All steps were performed at room temperature, with the exception of the fact that the Hydroxyapatite product was chilled prior to loading. The equilibration buffer used was chosen to match the hydroxyapatite elution buffer.

TABLE 4
Multimodal Capto Adhere processing steps

| Step | Buffer | Length of Step |
|---|---|---|
| Sanitization | 0.5N NaOH | 4 CV |
| Equilibration | 50 mM MOPS, 50 mM KPi, pH 7 | 10 CV |
| Load | Hydroxyapatite product | n/a |
| Wash | 200 mm KPi, pH 7 | 15 CV |
| Elution (Step) | 500 mM KPi, 200 mM KCl, pH 7 | 25 CV |
| Strip | 50 mM MOPS, 1M NaCl, pH 6.5 | 20 CV |

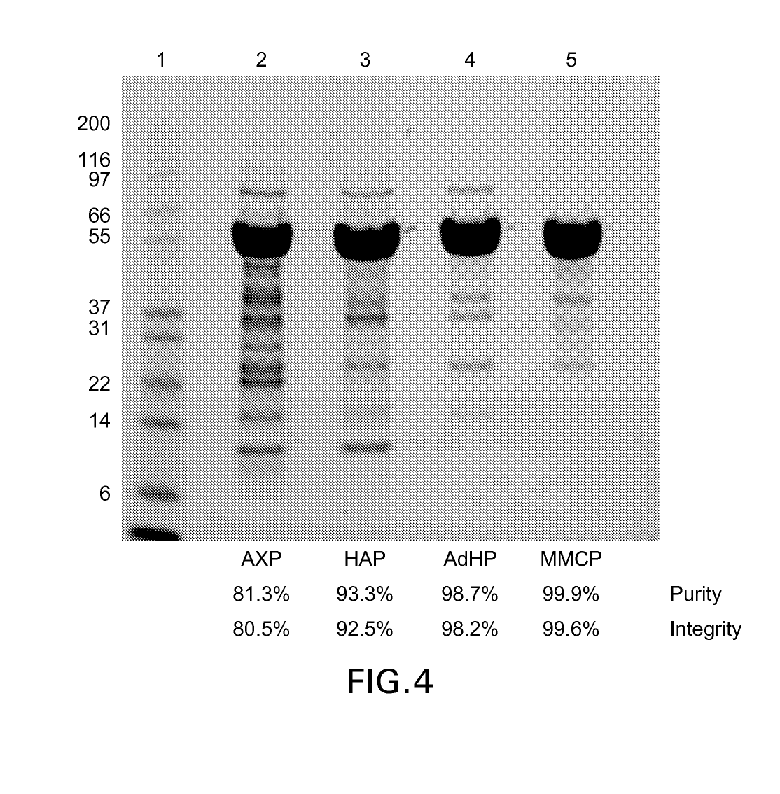

Purity results for the chromatography products are shown in FIG. 4. As shown, the purity was slightly higher for MMC product than Adhere product. The chromatography step yields for the two columns are 107% for Capto Adhere and 56% for Capto-MMC™. Concentrations used to calculate yields are by Capillary Isoelectric Focusing (multimodal feeds) and UV (Adhere/MMC products). The comparison makes clear that at least for the processing schemes used in this batch, the choice between MMC and Adhere comes down to yield versus purity. A 4-12% Bis-Tris NuPAGE gel was used with lx MES running buffer.

What is claimed is:

1. A method of purifying diptheria toxin, or a mutant form thereof, from a mixture containing the diptheria toxin, or mutant form thereof, comprising:
a) contacting the mixture with a first separation agent under conditions such that the diphtheria toxin, or mutant form thereof, binds to the first separation agent;
b) eluting the diphtheria toxin, or mutant form thereof, from the first separation agent;
c) contacting the eluted material obtained from step a) with a second separation agent under conditions such that the diphtheria toxin, or mutant form thereof, binds to the second separation agent; and
d) eluting said diptheria toxin, or mutant form thereof, from the second separation agent;
wherein either 1) the first separation agent is hydroxyapatite and the second separation agent is a multimodal resin; or 2) the first separation agent is a multimodal resin and the second separation agent is hydroxyapatite; and when the first separation agent or second separation agent is hydroxyapatite, prior to eluting from the hydroxyapatite, the hydroxyapatite undergoes a washing step under conditions such that impurities are removed.

2. The method of claim 1, wherein the washing is with a wash solution comprising from 0.01 to 1.0 M potassium chloride or sodium chloride at a pH from 6.5 to 8.0.

3. The method of claim 2, wherein the wash buffer further comprises 0.1 to 20 mM potassium phosphate or sodium phosphate.

4. The method of claim 1, wherein said eluting from hydroxyapatite comprises
a) step elution with an elution buffer comprising ≧about 30 mM potassium chloride or sodium chloride or about ≧15 mM potassium phosphate or sodium phosphate;
b) gradient elution comprising from about 10 to about 25 mM potassium phosphate or sodium phosphate or from about 100 mM to 2 M potassium chloride or sodium chloride; or
c) a pH change of ≧0.3 pH units.

5. The method of claim 1, wherein the mixture or eluted material contacted with the multimodal resin comprises Tris, MES, MOPS, HEPES, phosphate, acetate, chloride or sulfate.

6. The method of claim 1, wherein said eluting from the multimodal resin comprises
a) step elution with an elution buffer comprising ≧about 125 mM potassium chloride or sodium chloride at pH 6.8 to 9.5;
b) gradient elution comprising from about 0.2 to about 0.3 M sodium chloride, potassium chloride, sodium sulfate, ammonium sulfate, lithium sulfate, lithium chloride, or ammonium chloride;
c) a pH change of ≧0.5 pH units within a pH range of 6.5 to 9.5; or
d) a temperature change of ≧1° C. within a temperature of 2 to 30° C.

7. The method of claim 5, wherein the mixture or eluted material contacted with the multimodal resin comprises EDTA or a protease inhibitor.

8. The method of claim 6, wherein said eluting occurs in the presence of EDTA or a protease inhibitor.

9. The method of claim 1, wherein the first separation agent is hydroxyapatite and the second separation agent is a multimodal resin.

10. The method of claim 1, wherein the multimodal resin contains ligands which comprise a charged part and a hydrophobic part.

11. The method of claim 10, wherein the charged part is a negatively charged part.

12. The method of claim 11, wherein said negatively charged part is an anionic carboxylate group or anionic sulfo group for cation exchange.

13. The method of claim 12, wherein said multimodal resin is Capto-MMC™.

14. The method of claim 10, wherein the charged part is a positively charged part.

15. The method of claim 14, wherein said multimodal resin is Capto Adhere™.

16. The method of claim 1, wherein, prior performing step (a), the mixture is subject to one or more of the following: centrifugation, flocculation, clarification, or anion-exchange chromatography.

17. The method of claim 16, wherein the mixture is subject to two or three passes on anion-exchange chromatography.

18. The method of claim 16, wherein the mixture is subject to centrifugation, flocculation, clarification and anion-exchange chromatography.

19. The method of claim 18, wherein said clarifying is through centrifugation and depth filtration.

20. The method of claim 1, wherein the mixture is obtained from cultured host cells.

21. The method of claim 20, wherein the cultured host cells are osmotically shocked.

22. The method of claim 20, wherein said fermentation cells are recovered by centrifugation or microfiltration.

23. The method of claim 1, wherein the mixture is obtained from a cell-free production system.

24. The method of claim 1, wherein the diphtheria toxin, or mutant form thereof, is subject to one or more of the following step (d): centrifugation, ultrafiltration, microfiltration, filtration and anion-exchange membrane chromatography.

25. The method of claim 24, wherein the diphtheria toxin, or mutant form thereof, is subject to ultrafiltration, microfiltration, filtration and anion-exchange membrane chromatography.

26. The method of claim 1, wherein the mutant diphtheria toxin is CRM₁₉₇.

27. A formulation comprising the diptheria toxin, or a mutant form thereof, obtained according to the method of claim 1 and in liquid form.

28. The formulation of claim 27, wherein the purity of the diptheria toxin, or a mutant form thereof, is greater than 90% when maintained at a concentration of 10 g/L or more at 2° C. for at least six months.
